(19) United States
(12) Plant Patent Application Publication
(10) Pub. No.: US 2002/0166152 P1
(43) Pub. Date: Nov. 7, 2002

(54) Caryopteris plant named 'Inoveris'

(76) Inventor: M. Alain Cadic, Beaucouze (FR)

Correspondence Address: BURNS DOANE SWECKER & MATHIS L L P, POST OFFICE BOX 1404, ALEXANDRIA, VA, 22313-1404, US

(21) Appl. No.: 09/791,738

(22) Filed: Feb. 26, 2001

(51) Int. Cl. A01H 5/00

(52) U.S. Cl. PLT/226

(57) ABSTRACT

A new and distinct cultivar of Caryopteris×clandonensis plant is provided that is a seedling formed following the induced mutation of the ‘Heavenly Blue’ cultivar (U.S. Plant Pat. No. 1,091). Gamma radiation was utilized to create the mutation. The new cultivar can be readily distinguished from its parent by a more upright and more uniform growth habit, darker green foliage, more intense violet-blue flowers, and shorter internodes.

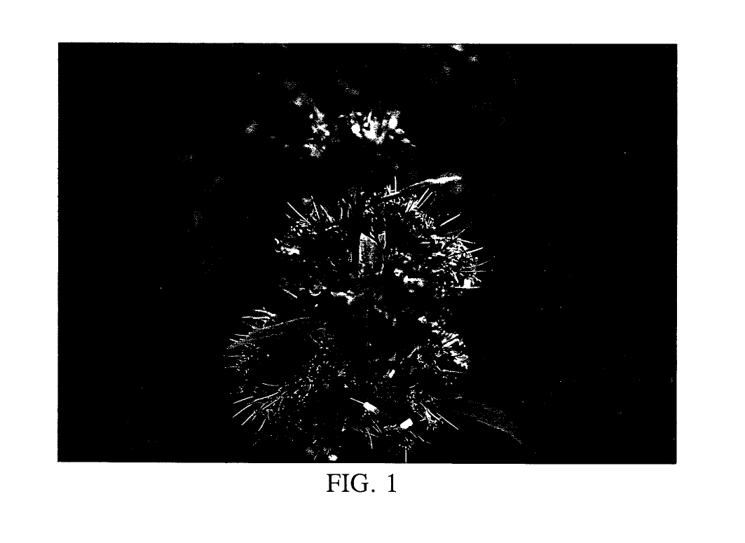

### SUMMARY OF THE INVENTION

A new and distinct cultivar of Caryopteris×clandonensis plant is provided. Caryopteris plants are generally recognized to be a hybrid of Caryopteris incana×Caryopteris mongholica and to be a better garden plant than either parent.

The new cultivar of the present invention is a mutation derived from the ‘Heavenly Blue’ cultivar (U.S. Plant Pat. No. 1,091). When creating the new cultivar, plants of the ‘Heavenly Blue’ cultivar were irradiated with cobalt gamma rays, seeds were formed thereon following self-pollination, and seedlings were produced upon planting which were observed and studied.

The irradiation and the study of the resulting seedlings were carried out at the Institut National De La Recherche Agronomique located at Angers, France.

It was found that a single plant observed following such irradiation and the planting of seeds possessed the characteristics of the Caryopteris cultivar of the present invention. The characteristics of such new cultivar can be summarized as follows when compared to the parent ‘Heavenly Blue’ cultivar:

(a) forms a more upright and more uniform growth habit,

(b) displays darker green foliage,

(c) forms in profusion attractive flowers that are a more intense violet-blue, and

(d) forms shorter internodes.

The new cultivar of the present invention well meets the needs of the horticultural industry, and is particularly well suited for growing as attractive ornamentation in the landscape. It can be treated as a perennial in the garden, and gives nice coloration in the garden during late summer.

As indicated, the new cultivar can be readily distinguished from its ‘Heavenly Blue’ parent.

The new cultivar has been found to readily undergo asexual propagation by the use of cuttings. Such asexual propagation has been carried out at Angers, France, and has confirmed that the characteristics are firmly fixed and are transmitted from one generation to another.

The new cultivar has been named the ‘Inoveris’ cultivar.

### BRIEF DESCRIPTION OF THE PHOTOGRAPH

The accompanying photograph shows typical blossoms and foliage of the new variety. The depicted plant was photographed on August 1st while growing outdoors at Angers, France. The attractive intense violet-blue blossoms and the nicely contrasting green foliage are illustrated.

### DETAILED DESCRIPTION

The following description is based on the observation of plants of the new cultivar growing outdoors at Angers, France. Such plants had been asexually reproduced through the use of softwood cuttings. The color terminology utilized in the description that follows is to be accorded its ordinary dictionary significance. Reference to the R.H.S. Colour Chart of The Royal Horticultural Society, London, England, sometimes is included.

| ORIGIN: | Seedling produced following the induced mutation | |
|---|---|---|
| | of the ‘Heavenly Blue’ cultivar (United States | |
| | Plant Patent 1,091). The ‘Heavenly Blue’ cultivar | |
| | was irradiated with gamma rays from cobalt 60. | |
| PARENTAGE: | Seedling of ‘Heavenly Blue’. | |
| CLASSIFICATION: | Caryopteris x clandonensis cv. ‘Inoveris’. | |
| PLANT: | form | attractive flowering shrub. |
| | habit | more upright and more uniform than |
| | | the ‘Heavenly Blue’ cultivar. The |
| | | habit is more regular and less random |
| | | appearing than that of the ‘Heavenly |
| | | Blue’ cultivar. |
| | internode | shorter than those of the parent |
| | length | ‘Heavenly Blue’ cultivar. |
| | configuration | dense and taller than it is broad. |
| | limbs | possess a bright aspect. |
| FOLIAGE: | disposition | opposite. |
| | configuration | lanceolate. |
| | margins | generally entire, but with a few teeth |
| | | that appear at broad intervals. |
| | color | dark green and darker than the |
| | | ‘Heavenly Blue’ parent. |
| INFLORESCENCE: | bearing | borne in profusion in cymes as illus- |
| | | trated. |
| | time of | July to October at Angers, France. |
| | bearing | |
| | color | dark violet-blue as illustrated, Violet- |
| | | Blue Group 93A, with some lighting |
| | | as the blossoms mature. The bloom |
| | | coloration is more intense than that |
| | | of the ‘Heavenly Blue’ cultivar. |
| GROWING | | Does well in full to moderate |
| CONDITIONS: | | sun, and prefers well-drained soil. |
| PROPAGATION: | | Can be readily propagated while uti- |
| | | lizing cuttings. |
| USAGE: | | Provides attractive ornamentation |
| | | when in bloom during late summer |
| | | when few other woody plants bloom. |
| | | Can be grown in the ground or in |
| | | containers. |

I claim:

1. A new and distinct cultivar of Caryopteris×clandonensis plant derived from the ‘Heavenly Blue’ cultivar (U.S. Plant Pat. No. 1,091) having the following combination of characteristics when compared to its ‘Heavenly Blue’ parent:
(a) forms a more upright and more uniform growth habit,
(b) displays darker green foliage,
(c) forms in profusion attractive flowers that are a more intense violet-blue, and
(d) forms shorter internodes;
substantially as described and illustrated.
